(19) United States
(12) Patent Application Publication
(10) Pub. No.: US 2021/0277189 A1
(43) Pub. Date: Sep. 9, 2021

(54) BIOBASED SUPERABSORBENT HYDROGELS

(71) Applicant: The United States of America, as Represented by the Secretary of Agriculture, Washington, DC (US)

(72) Inventors: VEERA M. BODDU, PEORIA, IL (US); JINGYUAN XU, DUNLAP, IL (US); GEORGE F. FANTA, MORTON, IL (US)

(21) Appl. No.: 16/811,016

(22) Filed: Mar. 6, 2020

(51) Int. Cl. C08J 3/075; C09K 17/32; A61K 47/38; C08J 3/24; A61K 9/20; A61K 9/48; B01J 20/24; B01J 20/28; B01J 20/30

(52) U.S. Cl. CPC C08J 3/075; C09K 17/32; A61K 47/38; C08J 3/247; A61K 9/2054; C08J 2401/28; B01J 20/24; B01J 20/28047; B01J 20/3085; C08J 2301/28; A61K 9/4866

(57) ABSTRACT

The present invention relates to superabsorbent biobased hydrogels prepared with sodium carboxymethyl cellulose (CMCNa) and hydroxyethyl cellulose (HEC) cross-linked with either citric acid, succinic acid, or sebacic acid. The swelling ratio of the superabsorbent hydrogels of the invention was dependent on the cross-linker used, the concentration of the cross-linker, and the cross-linking temperature.

### FIELD OF THE INVENTION

This invention relates to biobased superabsorbent polymer hydrogels, methods to prepare such biobased superabsorbent polymer hydrogels, and uses for such biobased superabsorbent polymer hydrogels.

### BACKGROUND OF THE INVENTION

Hydrogel research and development attracted much interest within recent decades for applications in drug delivery, tissue engineering, soil conditioning (Guiherme M. R. et al., 2015, “Superabsorbent hydrogels based on polysaccharide, for application in agriculture as soil conditioner and nutrient carrier—A review” Eur. Pol. J. 72: 365-385), and other non-hygienic utilizations (Cabo E. and Khutoryanskiy V. V., 2015, “Biomedical applications of hydrogels: A review of patents and commercial products,” Eur. Pol. J. 65: 252-267). Superabsorbent hydrogels are unique because they can absorb amounts of water that are much greater than their dry weight. Most commonly used superabsorbent agents are polyacrylates and copolymers with polyacrylates (Fekete T. et al., 2017, “Synthesis of carboxymethylcellulose/starch superabsorbent hydrogels by gamma-irradiation,” Chem. Cent. J., DOI 10.1186/s13065-017-0273-5). Recently, scientists seek greener and less expensive materials such as cellulose, starch, and their derivatives for the preparation of superabsorbent hydrogels (Chang C. and Zhang L., 2011, “Cellulose-based hydrogels: Present status and application prospects. Carbohydrate Polymers,” Carbohydr. Polym. 84: 40-53). Hydrogels are used at least for manufacturing contact lenses, hygiene products, tissue engineering scaffolds, drug delivery systems, wound dressings, well-coatings for cell culture, biosensors, electrocardiogram and electroencephalogram medical electrodes, water gel explosives, soil conditioning, soil nutrient carriers, soil erosion reduction, and improving water infiltration of fine textured soils.

Cellulose is the most abundant of the natural biopolymers existing on earth. It can be obtained from many sources, such as crop plants, wood, cotton, and agricultural wastes, as well as from non-plant resources (Xu J. et al., 2018, “Production and characterization of cellulose nanofibril (CNF) from agricultural waste corn stover,” Carbohydr. Polym. 192:202-207). Due to its plentiful, inexpensive, and renewable nature, cellulose can become the major chemical resource in the future (Chang C. and Zhang L., 2011, “Cellulose-based hydrogels: Present status and application prospects,” Carbohyrd. Polym. 84: 40-53). Many hydroxyl groups of cellulose make it relatively easy to prepare hydrogels. However, due to cellulose's insolubility in water, cellulose hydrogels are made mostly from cellulose derivatives (Fekete T. et al., 2017). Hydrogels may be prepared from different cellulose derivatives including methyl cellulose (MC), carboxymethyl cellulose (CMC), hydroxyethyl cellulose (HEC), hydroxypropyl cellulose (HPC), and hydroxypropylmethyl cellulose (HPMC) (Chang C. and Zhang L., 2011). By cross-linking CMC and HEC with divinylsulfone (DVS), Marci G. et al. prepared superabsorbent hydrogels (2006, “Environmentally sustainable production of cellulose-based superabsorbent hydrogels,” Green Chem. 8: 439-444). Demitri et al. reported the development of cellulose-based superabsorbent hydrogels by cross-linking CMC and HEC with citric acid (2008, “Novel superabsorbent cellulose-based hydrogels cross-linked with citric acid,” J. App. Polymer Sci. 110: 2453-2460).

U.S. Pat. No. 8,658,147 discloses superabsorbent polymer hydrogels prepared with a mixture of carboxymethyl cellulose and hydroxyethyl cellulose cross-linked with citric acid. The samples were pre-dried at 30° C. for 24 hours, and kept for 24 hours at 80° C. or 120° C. for the cross-linking reaction to occur. Citric acid was used at 1.75%; 2.75%; 3.75%; 10%; and 20% to obtain samples with different degrees of cross-linking.

There exists a need for inexpensive, superabsorbent biobased hydrogels with different swelling ratios, that can be used for various applications.

### SUMMARY OF THE INVENTION

Provided herein are biobased polymer hydrogels, methods to prepare such polymer biobased hydrogels, and uses for such polymer biobased hydrogels.

In an embodiment, the invention relates to polymer hydrogels comprising sodium carboxymethyl cellulose (CMCNa) and hydroxyethyl cellulose (HEC) covalently cross-linked with citric acid, succinic acid, or sebacic acid. In some embodiments of the invention, the polymer hydrogel comprises a 3 to 1 ratio of CMCNa to HEC. In some embodiments of the invention, the polymer hydrogel is cross-linked with citric acid. In some embodiments of the invention, the polymer hydrogel is cross-linked with 0.75%; 1.00%; 1.75%; or 2.75% citric acid. In some embodiments of the invention, the polymer hydrogel is cross-linked with succinic acid. In some embodiments of the invention, the polymer hydrogel is cross-linked with 0.75%; 1.00%; 1.75%; or 2.75% succinic acid. In some embodiments of the invention, the polymer hydrogel is cross-linked with sebacic acid. In some embodiments of the invention, the polymer hydrogel is cross-linked with 1.75%, 2.75%, 3.50%, or 4.255% sebacic acid.

In an embodiment, the invention relates to a polymer hydrogel that can absorb at least about 10 times its dry weight of water at room temperature. In some embodiments of the invention, the polymer hydrogel can absorb at least about 50 times its dry weight of water at room temperature. In some embodiments of the invention, the polymer hydrogel can absorb at least about 25 times its dry weight of water at room temperature. In some embodiments of the invention, the polymer hydrogel can absorb at least about 80 times its dry weight of water at room temperature.

In an embodiment, the invention relates to a composition comprising at least one polymer hydrogel comprising CMCNa and HEC covalently cross-linked with citric acid, succinic acid, or sebacic acid. In some embodiments of the invention, the composition is a contact lens, a hygiene product, a tissue engineering scaffold, a drug delivery system, a wound dressing, a cell culture well-coating, a biosensor, a medical electrode, a water gel explosive, a soil conditioning element, a soil nutrient carrier, a soil erosion reduction element, or a water infiltration soil additive. In some embodiments, the invention relates to a pharmaceutical composition comprising at least one polymer hydrogel comprising CMCNa and HEC covalently cross-linked with citric acid, succinic acid, or sebacic acid, and optionally a pharmaceutically-acceptable carrier. In some embodiments of the invention, the pharmaceutical composition is in the form of a tablet or a capsule.

In an embodiment, the invention relates to a method for preparing a polymer hydrogel. The method comprises mixing CMCNa, HEC, and citric acid, succinic acid, or sebacic acid to form a reaction solution; concentrating the reaction solution by heating to remove water; and heating the concentrated reaction solution to cross-link the hydrogel. In some embodiments of the invention, the CMCNa and HEC are used in a 3:1 ratio in the method for preparing a polymer hydrogel. In some embodiments of the invention, citric acid or succinic acid are added at 0.75%; 1.00%; 1.75%; or 2.75%. In some embodiments of the invention, sebacic acid is added at 1.75%; 2.75%; 3.50%; or 4.25%. In some embodiments of the invention, concentrating the reaction solution by heating to remove water is performed by heating for at least about 8 hours to about 18 hours. In some embodiments of the invention, heating to remove water is performed at 55° C. In some embodiments of the invention, the concentrated solution is heated at 70° C.; 80° C.; 90° C.; or 100° C.

### BRIEF DESCRIPTION OF THE DRAWINGS

FIG. 1A to FIG. 1C depict the molecular structures of the cross-linking reagents used. FIG. 1A shows citric acid; FIG. 1B shows succinic acid; FIG. 1C shows sebacic acid.

FIG. 2 depicts a graph of the swelling ratio of cellulosic hydrogel cross-linked with different amounts of citric acid at 90° C. for 24 hours. The Y axis depicts the swelling ratio in grams per gram (g/g); the X axis depicts the time in hours that the hydrogel was immersed in water. Open circles show results for hydrogel cross-linked with 0.75% citric acid; filled circles show results for hydrogel cross-linked with 1.00% citric acid; open squares show results for hydrogel cross-linked with 1.75% citric acid; filled squares show results for hydrogel cross-linked with 2.75% citric acid.

FIG. 3 depicts a graph of the swelling ratio of cellulosic hydrogel cross-linked with 1.75% citric acid for 24 hours at different temperatures. The Y axis depicts the swelling ratio in grams per gram (g/g); the X axis depicts reaction temperatures in ° C.

FIG. 4 depicts a graph of the swelling ratio of cellulosic hydrogel cross-linked with different amounts of succinic acid for 24 hours at 90° C. The Y axis depicts the swelling ratio in grams per gram (g/g); the X axis depicts the time in hours that the hydrogel was immersed in water. Open circles show results for hydrogel cross-linked with 0.75% succinic acid; filled circles show results for hydrogel cross-linked with 1.00% succinic acid; open squares show results for hydrogel cross-linked with 1.75% succinic acid; filled squares show results for hydrogel cross-linked with 2.75% succinic acid.

FIG. 5 depicts a graph of the swelling ratio of cellulosic hydrogel cross-linked with different amounts of sebacic acid at 90° C. for 24 hours. The Y axis depicts the swelling ratio in grams per gram (g/g); the X axis depicts the time in hours that the hydrogel was immersed in water. Open circles show results for hydrogel cross-linked with 1.75% sebacic acid; filled circles show results for hydrogel cross-linked with 2.75% sebacic acid; open squares show results for hydrogel cross-linked with 3.50% sebacic acid; filled squares show results for hydrogel cross-linked with 4.25% sebacic acid.

FIG. 6A to FIG. 6G depict graphs of the weight loss (wt) and/or differential thermogravimetric weight loss (DTG) of the polysaccharides, cross-linkers, and hydrogels of the invention. FIG. 6A shows the wt and DTG for CMCNa alone; FIG. 6B shows the wt and DTG for HEC; FIG. 6C shows the wt and DTG for a 3:1 mixture of CMCNa:HEC; FIG. 6D shows the wt for (a) citric acid, (b) succinic acid, and (c) sebacic acid; FIG. 6E shows the wt for the hydrogels of the invention formed by cross-linking a 3:1 mixture of CMCNa:HEC with (a) citric acid, (b) succinic acid, or (c) sebacic acid; FIG. 6F shows an expanded view of the wt between 200° C. and 250° C. from the graph shown in FIG. 6E; FIG. 6G shows the DTG for the hydrogels of the invention formed by cross-linking a 3:1 mixture of CMCNa:HEC with (a) citric acid, (b) succinic acid, or (c) sebacic acid. The X axis presents the temperature in ° C. The Y axis presents the percent weight loss (wt %), or the percent DTG (d(Wt %)/dT.

### DETAILED DESCRIPTION

The inventors have prepared cellulose derivative hydrogels from sodium carboxymethyl cellulose (CMCNa) and hydroxyethyl cellulose (HEC) using citric acid, succinic acid, or sebacic acid as cross-linker. The inventors studied the water absorption properties of the hydrogels against the three different cross-linkers, the concentration of the cross-linkers, and the cross-linking reaction temperature.

The present invention provides polymer hydrogels, methods of preparing the polymer hydrogels, methods of using the polymer hydrogels, and articles of manufacture comprising the polymer hydrogels.

Carboxymethylcellulose sodium salt (CMCNa) is a beta-(1,4)-D-glucopyranose polymer cellulose derivative with carboxymethyl groups bound to some of the hydroxyl groups of the glucopyranose monomers that make up the cellulose backbone. CMCNa is used as a bulk laxative, as an emulsifier and thickener in cosmetics and pharmaceuticals, and as a reagent stabilizer.

Hydroxyethyl cellulose (HEC) is a gelling and thickening agent derived from cellulose. It is widely used in cosmetics, cleaning solutions, and other household products.

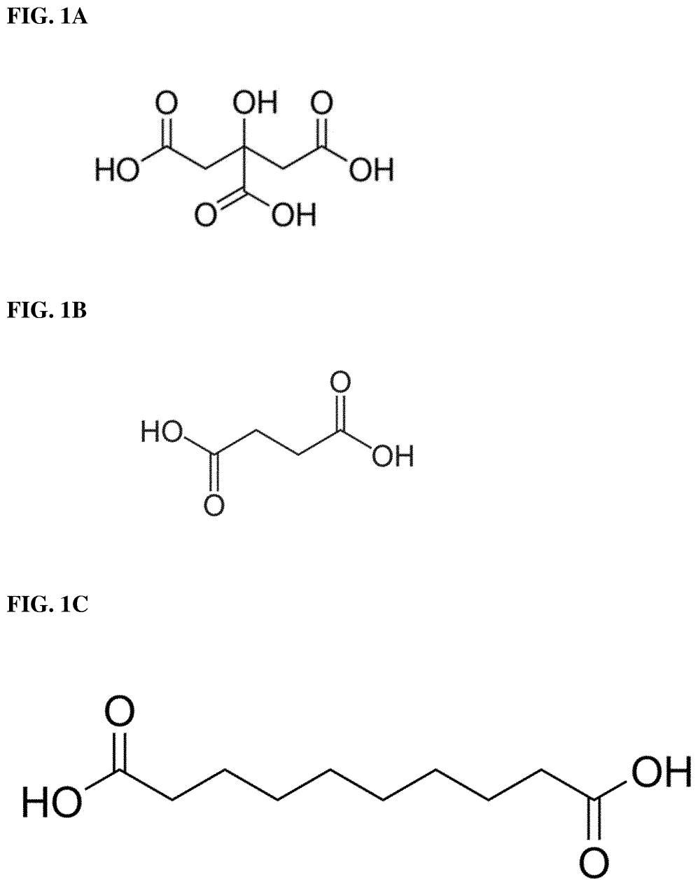

Citric acid is a tricarboxylic acid with the molecular formula CH₂COOH—C(OH)COOH—CH₂COOH, and the chemical structure as depicted in FIG. 1C. Citric acid is a weak organic acid found naturally in citrus fruits, especially lemons and limes. Citric acid is used as an excipient in pharmaceutical preparations, as an acidulant to control pH, and acts as an anticoagulant by chelating calcium in blood.

Succinic acid is a dicarboxylic acid with the molecular formula (CH₂)₂(CO₂H)₂, and the chemical structure as depicted in FIG. 1B. Succinate, the succinic acid anion, is a component of the citric acid cycle and is capable of donating electrons to the electron transfer chain. Succinic acid is a colorless crystal with an acid taste that is used as a chemical intermediate in medicine, the manufacture of lacquers, and in the preparation of perfume esters. Succinic acid is used in foods as a sequestrant, buffer, and neutralizing agent.

Sebacic acid is a dicarboxylic acid with the molecular formula (CH₂)₈(CO₂H)₂, and the chemical structure as depicted in FIG. 1C.

In an embodiment, the invention relates to polymer hydrogels comprising CMCNa and HEC covalently cross-linked with citric acid, succinic acid, or sebacic acid. In some embodiments of the invention, the polymer hydrogels comprise a 3 to 1 ratio of CMCNa to HEC. In some embodiments of the invention, the polymer hydrogels of the invention are cross-linked with from about 0.5% to about 3.0% citric acid. In some embodiments of the invention, the polymer hydrogels are cross-linked with 0.75%; 1.00%; 1.75%; or 2.75% citric acid. In some embodiments of the invention, the polymer hydrogels comprising CMCNa and HEC are cross-linked with citric acid at 90° C. for 24 hours.

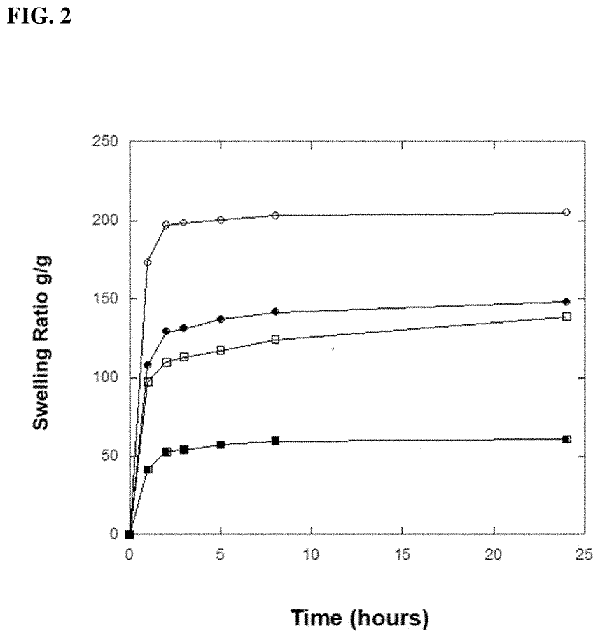

A graph of the swelling ratio of cellulosic hydrogel cross-linked with different amounts of citric acid at 90° C. for 24 hours is shown in FIG. 2. Open circles show results for hydrogel cross-linked with 0.75% citric acid; filled circles show results for hydrogel cross-linked with 1.00% citric acid; open squares show results for hydrogel cross-linked with 1.75% citric acid; filled squares show results for hydrogel cross-linked with 2.75% citric acid. This figure shows that the amount of water absorbed by polymer hydrogels cross-linked with 0.75%; 1.00%; 1.75%; or 2.75% citric acid at 90° C. for 24 hours increased with the length of time that the sample was immersed in water, and increased as the amount of cross-linker increased. Hydrogels cross-linked with 0.75%; 1%; and 2.75% citric acid at 90° C. for 24 hours became saturated after several hours, while hydrogels cross-linked with 1.75% citric acid at 90° C. for 24 hours did not appear to saturate even after 24 hours. After being immersed in water for 24 hours, hydrogels cross-linked with 0.75% citric acid absorbed about 205 times their own weight of water; hydrogel cross-linked with 1.00% citric acid absorbed about 148 times their own weight of water; hydrogels cross-linked with 1.75% citric acid absorbed only about 139 times their own weight of water; and hydrogels cross-linked with 2.75% citric acid absorbed only about 61 times their own weight of water.

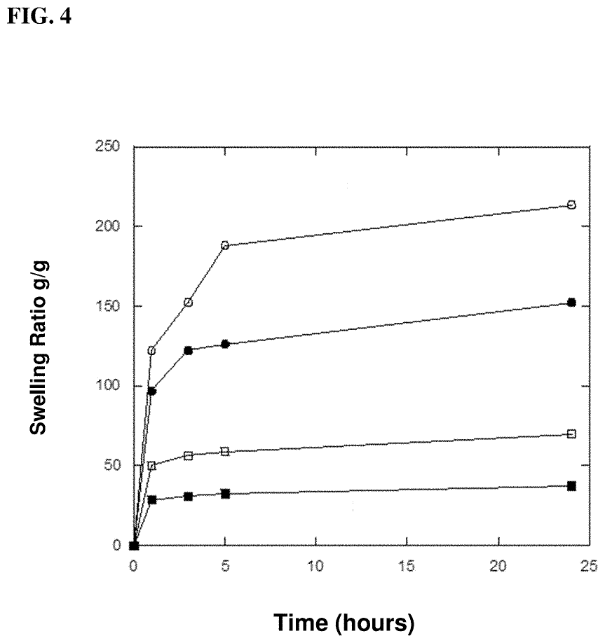

In an embodiment, the invention relates to polymer hydrogels cross-linked with succinic acid. In some embodiments of the invention, the polymer hydrogels are cross-linked with from about 0.5% to about 3.0% succinic acid. In some embodiments of the invention, the polymer hydrogels are cross-linked with 0.75%; 1.00%; 1.75%; or 2.75% succinic acid. A graph of the swelling ratio of polymer hydrogels cross-linked with different amounts of succinic acid for 24 hours at 90° C. is shown in FIG. 4. Open circles show results for hydrogel cross-linked with 0.75% succinic acid; filled circles show results for hydrogel cross-linked with 1.00% succinic acid; open squares show results for hydrogel cross-linked with 1.75% succinic acid; filled squares show results for hydrogel cross-linked with 2.75% succinic acid. After immersion in water for 24 hours, polymer hydrogels cross-linked with succinic acid show similar swelling ratios as polymer hydrogels cross-linked with citric acid. As seen in FIG. 4, the water absorption of polymer hydrogels cross-linked with succinic acid decreased with increased cross-linker concentrations, which was the same trend seen in FIG. 2 for polymer hydrogels cross-linked with citric acid. After immersion in water for 24 hours, polymer hydrogels cross-linked with 0.75% succinic acid absorbed more than 213 times their own weight of water. This result is similar to the swelling ratio of about 205 obtained with hydrogels cross-linked with 0.75% citric acid. Hydrogels cross-linked with 1.00% succinic acid absorbed more than 152 times their own weight of water. This result is similar to the swelling ratio of about 148 obtained for hydrogels cross-linked with 1.00% citric acid. Hydrogels cross-linked with 1.75%; or 2.75% succinic acid absorbed about half as much water than did hydrogels cross-linked with 1.75%; or 2.75% citric acid. Even after 24 hours immersed in water, none of the hydrogels cross-linked with succinic acid appeared to be saturated.

Citric acid and succinic acid similar molecular size, and have oxygen double bonds (see FIG. 1A and FIG. 1B), which are possible reaction sites for cross-linking with cellulose (Demitri, et al. 2008, supra). The citric acid chemical structure shown in FIG. 1A has three possible reaction site, while the succinic acid chemical structure shown FIG. 1B in has two sites. Therefore, the amount of water absorption of polymer hydrogels cross-linked with 0.75% citric acid will be similar to that of polymer hydrogels cross-linked with 0.75% succinic acid, and the amount of water absorption of polymer hydrogels cross-linked with 1.00% citric acid will be similar to that of polymer hydrogels cross-linked with 1.00% succinic acid. These similarities are due to the analogous cross-linking produced by citric acid and succinic acid. It may be possible that the cross-linking is different in polymer hydrogels cross-linked with higher amounts of citric acid or succinic acid (1.75% and 2.75%).

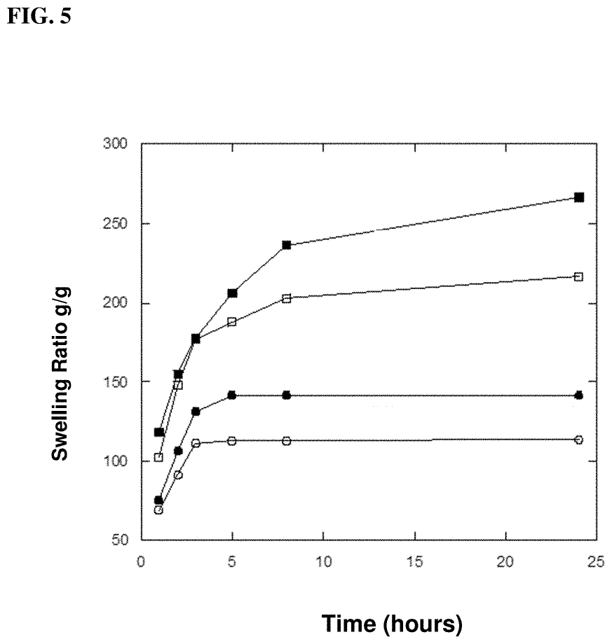

In an embodiment, the invention relates to polymer hydrogels cross-linked with sebacic acid. In some embodiments of the invention, the polymer hydrogels are cross-linked with from about 0.75% to about 5.0% sebacic acid. In some embodiments of the invention, the polymer hydrogels are cross-linked with 1.75%; 2.75%; 3.50%; or 4.25% sebacic acid. FIG. 5 depicts a graph of the swelling ratio of cellulosic hydrogel cross-linked with different amounts of sebacic acid at 90° C. for 24 hours. Open circles show results for hydrogel cross-linked with 1.75% sebacic acid; filled circles show results for hydrogel cross-linked with 2.75% sebacic acid; open squares show results for hydrogel cross-linked with 3.50% sebacic acid; filled squares show results for hydrogel cross-linked with 4.25% sebacic acid. Hydrogels cross-linked with sebacic acid presented very different water absorption properties when compared with hydrogels cross-linked with citric acid or with succinic acid. No hydrogel was formed when attempting to cross-link the CMCNa/HEC mixture with sebacic acid concentrations lower than 1.75%. The material formed did not have any water absorption capacity, which was similar to water absorption by sebacic acid alone, indicating that no significant cross-linking occurred. As seen in FIG. 5, water absorption of hydrogels cross-linked with sebacic acid increased as the concentration of sebacic acid increased. This trend is opposite to the one seen for hydrogels cross-linked with citric acid or succinic acid. The swelling ratio of hydrogels cross-linked with 1.75% sebacic acid was about 110, which is similar to the swelling ratio of hydrogels cross-linked with 2.75% citric acid. After immersion in water for 24 hours, hydrogels cross-linked with 2.75% sebacic acid absorbed more than about 140 times their own weight of water. Hydrogels cross-linked with 3.50% sebacic acid absorbed more than 210 times their own weight of water. Hydrogels cross-linked with 4.25% succinic acid absorbed at least about 260 times their own weight of water. Even after 24 hours immersed in water, none of the hydrogels cross-linked with sebacic acid appeared to be saturated.

Like succinic acid, sebacic acid has two reaction sites, but it has a longer ‘arm,’ as can be seen when comparing FIG. 1A and FIG. 1C. It may be that sebacic acid acts as a cross-linker and as part of the polymer network due to its longer arm. When attempting to prepare a polymer hydrogel with a sebacic acid concentration lower than 1.75%, no polymer hydrogel was formed because the sebacic acid mainly acted as chains for a part of the whole network, but the whole network was not uniformly cross-linked. Because of the relatively long arm in sebacic acid, the cross-linking was relatively ‘loose’ so that polymer hydrogels were not firm, even when using higher concentrations of sebacic acid (up to 4.25%).

In an embodiment of the invention, during the preparation of the polymer hydrogels of the invention a reaction solution comprising CMCNa, HEC, and a cross-linking reagent is concentrated by heating. In some embodiments of the invention, during the preparation of the polymer hydrogels of the invention, the reaction solution is concentrated by heating at about 55° C. In some embodiments of the invention, during the preparation of the polymer hydrogels of the invention, the reaction solution is concentrated by heating for at least about 8 hours to about 18 hours. In some embodiments of the invention, during the preparation of the polymer hydrogels of the invention, the reaction solution is heated for a period of time to allow dryness of the reaction mixture.

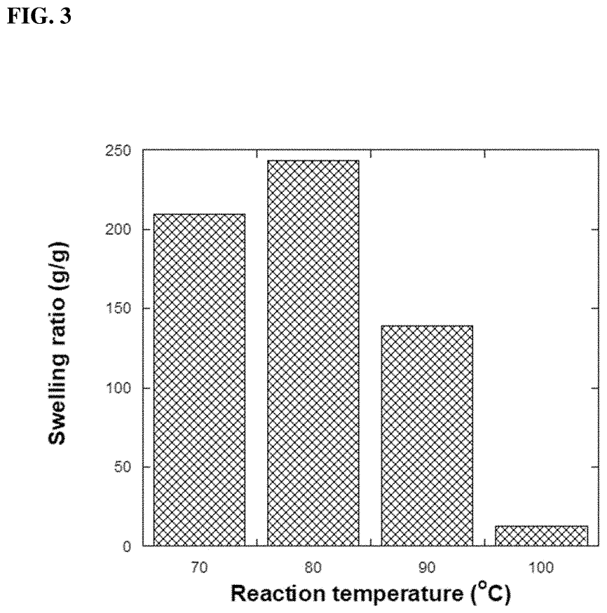

In an embodiment, the invention relates to polymer hydrogels prepared by cross-linking at elevated temperature. In some embodiments of the invention, during the preparation of the polymer hydrogels of the invention, after concentrating the reaction solution, the cross-linking reaction is conducted at elevated temperature, for example, at about 70° C.; about 80° C.; about 90° C.; or about 100° C. In some embodiments of the invention, the cross-liking reaction is conducted by heating the concentrated reaction solution for at least about 16 hours. FIG. 3 depicts a graph of the swelling ratio of polymer hydrogels cross-linked with 1.75% citric acid for 24 hours at different temperatures. As seen in FIG. 3, the temperature of the cross-linking reaction affected gel formation and water absorption. Hydrogels cross-linked with 1.75% citric acid at 70° C. behaved like soft solids, and were easily deformed. The swelling ratio of hydrogels cross-linked with 1.75% citric acid was about 210. Hydrogels cross-linked with 1.75% citric acid at 80° C. were slightly flabby and loose, and their swelling ratio reached about 243. Hydrogels cross-linked with 1.75% citric acid at 90° C. were not too firm or too soft, and their swelling ratio was about 138. Hydrogels cross-linked with 1.75% citric acid at 100° C. were very firm, and their swelling ratio dropped sharply to about 13.

In an embodiment, the invention relates to polymer hydrogels that can absorb at least about 25 times their dry weight of water at room temperature. In some embodiments of the invention, the polymer hydrogels are cross-linked with citric acid, and can absorb at least about 50 times its dry weight of water at room temperature. In some embodiments of the invention, the polymer hydrogels are cross-linked with sebacic acid, and can absorb at least about 80 times their dry weight of water at room temperature. In some embodiments of the invention, the polymer hydrogels are cross-linked with succinic acid, and can absorb at least about 25 times their dry weight of water at room temperature.

In an embodiment, the invention relates to a method for preparing polymer hydrogels of the invention. The method comprises mixing CMCNa and HEC with citric acid, succinic acid, or sebacic acid to form a reaction solution; concentrating the reaction solution by heating to remove water; and heating the concentrated reaction solution to cross-link the hydrogel. In some embodiments of the invention, the CMCNa and HEC used for preparing polymer hydrogels are present in a 3:1 ratio. In some embodiments of the invention, in the method for preparing polymer hydrogels citric acid or succinic acid are added at 0.75%; 1.00%; 1.75%; or 2.75%. In some embodiments of the invention, in the method for preparing polymer hydrogels sebacic acid is added at 1.75%; 2.75%; 3.50%; or 4.25%. In some embodiments of the invention, in the method for preparing polymer hydrogels of the invention, concentrating the reaction solution by heating to remove water is performed by heating for at least about 8 hours to about 18 hours. In some embodiments of the invention, in the method for preparing polymer hydrogels of the invention, the heating to remove water is performed at 55° C. In some embodiments of the invention, in the method for preparing polymer hydrogels of the invention the cross-linking is performed by heating the concentrated solution 70° C.; 80° C.; 90° C.; or 100° C.

The method for preparing a polymer hydrogel of the invention can further include the step of purifying the polymer hydrogel, for example, by washing the polymer hydrogel in a polar solvent. A polar solvent may be water, or a polar organic solvent. A polar organic solvent may be an alcohol, such as methanol or ethanol, or a combination thereof. The polymer hydrogel when immersed in the polar solvent swells and releases any component, such as by-products or unreacted polycarboxylic acid that was not incorporated into the polymer network. Water is preferred as the polar solvent; purified water such as distilled water or water purified by reverse osmosis is still more preferred. The Volume of water required during this step to reach the maximum swelling degree of the gel, is approximately 10-fold to 20-fold greater than the initial volume of the gel itself. Taking into account the substantial amounts of water which would be involved during this step on an industrial scale, as well as the disposal and/or recycling of the washes, the importance of avoiding the presence of any toxic by-products in the synthetic process becomes evident. The polymer hydrogel washing step may be repeated more than once, optionally changing the polar solvent employed. For example, the polymer hydrogel can be washed with methanol or ethanol followed by purified water, with these two steps optionally repeated one or more times.

The method for preparing a polymer hydrogel of the invention can further include drying of polymer hydrogel. The drying step is carried out by immersing the fully-swollen polymer hydrogel in a cellulose nonsolvent, a process known as phase inversion. Suitable cellulose non-solvents include, for example, acetone and ethanol. Drying the polymer hydrogel by phase inversion results in a final microporous structure which improves the absorption properties of the polymer hydrogel by capillarity. Moreover, if the porosity is interconnected or open, i.e. the micropores communicate with one another, the absorption/desorption kinetics of the polymer hydrogel will be improved as well. When a completely or partially swollen polymer hydrogel is immersed into a nonsolvent, the polymer hydrogel undergoes phase inversion with the expulsion of water, until the polymer hydrogel precipitates in the form of a vitreous solid as white-colored particles. Various rinses in the non-solvent may be necessary in order to obtain the dried polymer hydrogel in a short period of time. For example, when a swollen polymer hydrogel is immersed in acetone as the non-solvent, a water/acetone mixture is formed which increases in water content as the polymer hydrogel dries; at a certain acetone/water concentration, for example, about 55% in acetone, water is no longer able to exit from the polymer hydrogel, and thus fresh acetone has to be added to the polymer hydrogel to proceed with the drying process. The higher the acetone/water ratio during drying, the faster is the drying process. Pore dimensions are affected by the rate of the drying process and the initial dimensions of the polymer hydrogel particles: larger particles and a faster process tend to increase the pore dimensions; pore dimensions in the microscale range are preferred, as pores in this size range exhibit a strong capillary effect, resulting in the higher sorption and water retention capacity.

The polymer hydrogels of the invention can also be dried by a process such as air drying, freeze drying, or oven drying. These drying methods can be used alone, in combination, or in combination with the non-solvent drying step described above. For example, the polymer hydrogel can be dried in a non-solvent, followed by air drying, freeze drying, oven drying, or a combination thereof to eliminate any residual traces of non-solvent. Oven drying can be carried out at a temperature of approximately 30-45° C. until the residual nonsolvent is completely removed. The washed and dried polymer hydrogel can then be used as is, or can be milled to produce polymer hydrogel particles of a desired size.

Preparation of the polymer hydrogels of the invention does not include a compound that serves as a molecular spacer. The cross-linking solution can optionally include a compound which serves as a molecular spacer. Using a molecular spacer in the cross-linking solution allows for cross-linking to occur at sites which are not close together. This enhances the ability of the polymer network to expand so as to greatly increase the polymer hydrogel absorption properties. Compounds commonly used as molecular spacers include monosaccharides, disaccharides and sugar alcohols. The sugar alcohols may include sucrose, sorbitol, plant glycerol, mannitol, trehalose, lactose, maltose, erythritol, xylitol, lactitol, maltitol, arabitol, glycerol, isomalt, and cellobiose, among others. Molecular spacers are normally included in the cross-linking solution in the amount of about 0.5% to about 10% by weight relative to the solvent, more preferably about 2% to about 8%, and more preferably about 4%.

In an embodiment of the method of the invention, which results in the formation of superabsorbent polymer hydrogels having a particularly high swelling ratio (SR), the total precursor concentration in the aqueous solution is of at least 2% by weight referred to the weight of the water of the starting aqueous solution, and the amount of the cross-linking agent is between about 0.5% and about 5% by weight referred to the weight of the precursor. In the present description, the term “precursor” indicates CMCNa and HEC, the precursors used for the formation of the polymer hydrogel network. The aqueous solution containing the precursor does not include sorbitol.

The swelling ratio (SR) is a measure of the ability of the polymer hydrogel to absorb water. Calculation of SR is obtained through swelling measurements at the equilibrium (using, for example, a micro scale with a sensitivity of 10⁻⁵ g). SR is calculated using the following formula:

SR=(Wₛ−W_{d})/W_{d}

where W_{d} is the weight of the polymer hydrogel after drying to remove any residual water, and before immersion in distilled water; Wₛ is the weight of the polymer hydrogel after immersion in distilled water for different amounts of time.

In an embodiment, the cross-linking reaction to prepare the polymer hydrogels of the invention, is preferably carried out at a temperature between about 60° C. and 120° C. Varying the temperature during this stage of the process will enable one to increase or decrease the cross-linking degree of the polymer network. Depending on the polymer hydrogel characteristics desired, a cross-linking temperature of about 80° C. may be preferred.

In an embodiment, the invention relates to a method for preparing a hydrogel of desired characteristics. The method comprises dissolving CMCNa and HEC in water at room temperature; adding a cross-linker; concentrating the aqueous solution heating at 55° C. for at least 8 hours to remove the water from the solution; and heating the concentrated solution at a temperature of at least 70° C. for at least 8 hours to induce the cross-linking reaction and formation of a polymer hydrogel.

The polymer hydrogel may optionally be washed three times with water over about a 24 hour period. The washed polymer hydrogel may optionally be immersed in acetone for about 24 hours to remove water. Further, the polymer hydrogel may be dried in an oven at 45° C. for at least about 5 hours. The dried polymer hydrogel may be milled to provide polymer hydrogel particles.

In an embodiment, the polymer hydrogels of the invention have swelling ratios of at least about 5. In some embodiments of the invention, the polymer hydrogels are Superabsorbent polymer hydrogels, for example, polymer hydrogels having an SR of at least about 10. In some embodiments of the invention, the polymer hydrogels have SRs at least about 20, at least about 30, at least about 40, at least about 50, at least about 60, at least about 70, at least about 80, at least about 90, or at least about 100. For example, in certain embodiments, the polymer hydrogels of the invention have SRs from about 10 to about 100, from about 20 to about 100, from about 30 to about 100, from about 40 to about 100, from about 50 to about 100, from about 60 to about 100, from about 70 to about 100, from about 80 to about 100, or from about 90 to about 100. In certain embodiments, the invention includes polymer hydrogels having SRs up to 150, 200, 250, 300, 330 or 350. In certain embodiments, the polymer hydrogels of the invention can absorb an amount of one or more bodily fluids, Such as blood, blood plasma, urine, intestinal fluid or gastric fluid, which is at least 10, 20, 30, 40, 50, 60, 70, 80, 90, or 100 times their dry weight. The ability of the polymer hydrogel to absorb bodily fluids can be tested using conventional means, including testing with samples of bodily fluids obtained from one or more subjects or with simulated bodily fluids, such as simulated urine or gastric fluid. In certain preferred embodiments, the polymer hydrogels can absorb significant amounts of a fluid prepared by combining one Volume of simulated gastric fluid (SGF) with eight volumes of water. SGF can be prepared using USP Test Solutions procedures which are known in the art. In some embodiments, the polymer hydro gels of the invention can absorb at least about 10, 20, 30, 40, 50, 60, 70, 80, 90, 100 or more times their dry weight of this SGF/water mixture.

The polymer hydrogels of the invention include cross linked polymers having varying extents of hydration. For example, the polymer hydrogels can be provided in a state of hydration ranging from a substantially dry or anhydrous state, such as a state in which from about 0% to about 5% of the polymer hydrogel by weight is water or an aqueous fluid, to states comprising a substantial amount of water or aqueous fluid, including up to a state in which the polymer hydrogel has absorbed a maximum amount of water or an aqueous fluid.

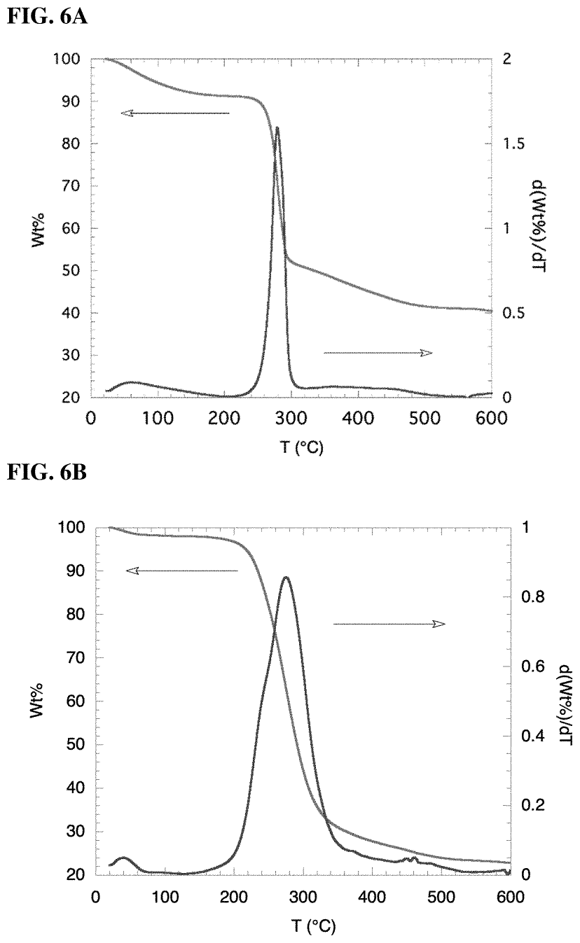

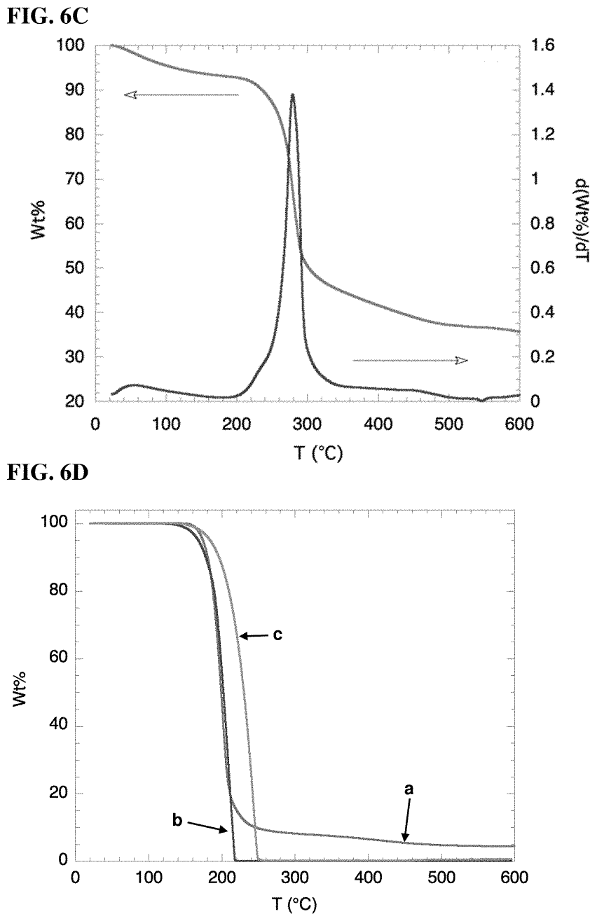

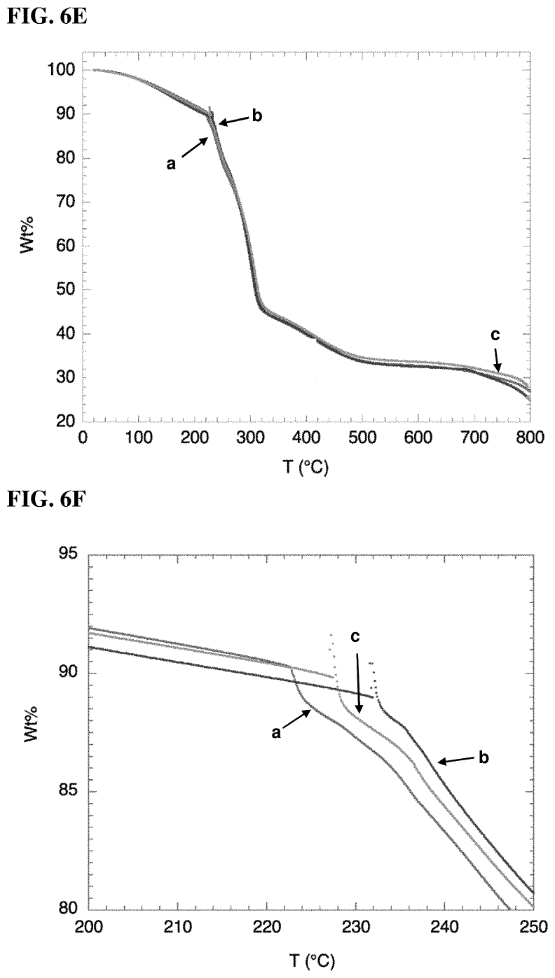

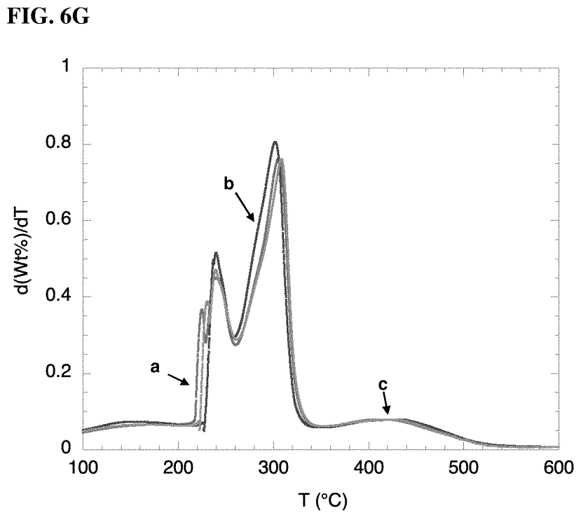

As an additional evidence for the cross-linking reaction on the polymer hydrogels of the invention, thermogravimetric analysis was performed. As shown in FIG. 6A, the major decomposition of CMCNa occurred at 250° C. to 300° C., and weight loss at 300° C. is about 50%. As seen on FIG. 6B, the major decomposition of HEC occurred at 200 C to 350 C, and the weight loss was about 85%. As seen on FIG. 6C, when CMCNa was mixed with HEC, the overall decomposition profile was similar to that of CMCNa alone. This may be because HEC was only 25% of the total reaction mix. As expected, and as seen on FIG. 6D, the weight loss of citric acid was very similar to that of succinic acid, and sebacic acid degraded slower. After reacting with each cross-linker (citric acid, succinic acid, or sebacic acid), the degradation profile of the CMCNa/HEC mixture was changed dramatically. As seen in FIG. 6F, unlike a single major decomposition of the starting material as seen on FIG. 6C, the cross-linked products showed two major peaks in the DTG. More specifically, the single peak at 290° C. seen with the CMCNa/HEC mixture before cross-linking was changed to two peaks, one at 240° C., and one at 300° C. to 310° C. This difference is not due to degradation of the cross-linkers because they are present at only 1.75% of the total weight of reaction mixture. And, the thermogram of each of the cross-linkers is different (see FIG. 6D). The shift of the decomposition peak position at 260 C to 330° C. to a higher temperature by about 20° C. after the reaction, indicates that the cross-linkers formed stronger bonds after the reaction than before the reaction. The minor DTG peaks shown in FIG. 7 are believed to be due to the small amount of gas generated at the beginning of the thermal degradation of the cross-linked products. When gas is generated, it exerts pressure on the TGA pan.

Because of their swelling ratios, the biobased hydrogels of the invention may be used in different applications such as agricultural seed coating, soil conditioning, soil nutrient carrier, soil erosion reduction element, or a water infiltration soil additive. Other possible uses are contact lenses, hygiene products, tissue engineering scaffolds, drug delivery systems, wound dressing, cell culture well-coatings, biosensors, medical electrodes, water gel explosives, paper pants/diaper materials, drug-delivery, wound-healing, or cosmetic materials. The biobased hydrogels of the invention may be in pharmaceutical compositions, optionally comprising a pharmaceutically-acceptable carrier. The pharmaceutical composition may be in the form of a tablet or a capsule.

The invention further includes compositions or articles of manufacture which comprise the polymer hydrogels of the invention. Such articles of manufacture include articles in which polyacrylic polymer hydrogels are conventionally used, in consumer products, such as for example absorbent products for personal care (i.e., diapers, sanitary towels, etc.) and in products for agriculture (e.g., devices for the controlled release of water and nutrients). The absorption properties of the polymer hydrogels of the invention, which in some embodiments depend on the amount and/or type of cross-linking material employed, are comparable to those of polyacrylic gels. The polymer hydrogels obtainable by the method of the present invention therefore possess mechanical properties which make them suitable for use in all of the above-mentioned fields. The polymer hydrogels of the invention, however, have advantages over acrylic polymer hydrogels, such as biodegradability, the absence of any toxic by-products during the manufacturing process, and the use of fewer and readily available reagents. Such features enable a use of the polymer hydrogels of the invention in the biomedical and pharmaceutical fields as well.

Thus, the scope of the present invention also includes the use of the polymer hydrogels obtainable by the method of the invention as an absorbent material in products which are capable of absorbing water and/or aqueous solutions and/or which are capable of swelling when brought into contact with water and/or an aqueous solution. The invention further includes the use of any of the polymer hydrogels of the invention in medicine. Such use includes the use of a polymer hydrogel in the preparation of a medicament for the treatment of obesity or any medical disorder or disease in which calorie restriction has a therapeutic, palliative or prophylactic benefit.

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

### EXAMPLES

Having now generally described this invention, the same will be better understood by reference to certain specific examples, which are included herein only to further illustrate the invention and are not intended to limit the scope of the invention as defined by the claims.

### Example 1

### Materials and Methods

The materials and methods used to prepare polymer hydrogels, determine their swelling ratio in water, and conduct thermogravimetric analysis of the polymer hydrogels follow.

Sodium carboxymethyl cellulose (CMCNa), hydroxyethyl cellulose (HEC), citric acid, succinic acid, and sebacic acid were all obtained from Sigma-Aldrich, St. Louis, Mo., USA; and used as received. The chemical structure of citric acid is depicted in FIG. 1A; the chemical structure of succinic acid is depicted in FIG. 1B; and the chemical structure of sebacic acid is depicted in FIG. 1C.

Polymer hydrogels were prepared following Demitri et al. (2008, “Novel superabsorbent cellulose-based hydrogels cross-linked with citric acid,” J. Appl. Polym. Sci. 110: 2453-2460) with some modifications. Briefly, a 2% (wt %) solution of a 3/1 weight ratio of CMCNa and HEC were combined using nanopure water at room temperature with continuous stirring until the solution became clear. Once the solution was clear, a cross-linker selected from citric acid, succinic acid, or sebacic acid was then added. Citric acid was added at 0.75%; 1.00%; 1.75%; or 2.75%; succinic acid was added at 0.75%, 1.00%, 1.75%, or 2.75%; sebacic acid was added at 1.75%; 2.75%; 3.50%; or 4.25%. The samples were then dried in an oven at 55° C. for about 12 to 18 hours to remove the water. Cross-linking was performed by heating the samples for 24 hours at 70° C.; 80° C.; 90° C.; or 100° C.

The attenuated total reflectance/Fourier transform infrared spectroscopy (ATR/FTIR) of the individual samples was measured as an indication of cross-linking and of a change in chemical composition.

The FTIR spectra of the individual samples were collected using a Thermo Fisher Nicolet iS10 FTIR spectrometer (Thermo Fisher Scientific, Waltham, Mass., USA) equipped with a SensIR dATR DuraScope single-contact attenuated total reflectance (ATR) attachment (Smith's Detection; Danbury, Conn., USA). The ATR attachment employed a 2 mm diameter round diamond crystal, a pressure gauge, and video imaging. The FTIR spectrometer was equipped with a Globar infrared source, KBr beam splitter, and a deuterated triglycine sulfate (DTGS) detector. The samples were freeze-dried and placed onto the surface of the diamond ATR crystal, pressure was applied, and spectra were acquired at room temperature in the region of 4000 cm⁻¹ to 700 cm⁻¹ (Seabourn B. W. et al., 2008, “Determination of secondary structural changes in gluten proteins during mixing using Fourier transform horizontal attenuated total reflectance spectroscopy,” J. Agr. Food Chem. 56: 4236-4243). A 128-scan co-added interferogram with Happ-Genzel apodization was employed for each sample at 4 cm⁻¹ resolution using OMNIC software (version 8.2; Thermo Fisher Scientific; Madison, Wis., USA). FTIR data was collected in an enclosed room with a single operator. A background scan was performed every 5 minutes.

Thermogravimetric analyses (TGA) were conducted on a Q500 TGA instrument (TA Instruments, New Castle, Del., USA). Approximately 5 mg of each sample were placed on a tared, open platinum TGA pan. The sample was heated from room temperature up to 800° C. under nitrogen at a rate of 10° C. per minute. Along with weight loss data, the differential thermogravimetric weight loss (DTG, %/° C.) was recorded as well. Each sample was run in duplicate.

The swelling ratio (SR) measurements followed the method described by Peppas N. A. (1987, “Hydrogels in medicine and pharmacy, properties and applications, N. A. Peppas (Ed.), Vol. 3, CRC Press, Boca Raton). Briefly, the SR was measured by determining the sample weight before and after the dry sample was submersed in water at room temperature. The SR was calculated as: SR=(Wₛ−W_{d})/W_{d}. Where Wₛ is the weight of the swollen hydrogel after submersion in water, and W_{d} is the weight of the dry sample.

### Example 2

### Swelling Ratio of Hydrogels Prepared with Citric Acid

This example shows that the swelling ratio of cellulose-based polymer hydrogels prepared with a CMCNa and HEC mixture cross-linked with citric acid for 24 hours at 90° C. is inversely proportional to the citric acid concentration used.

The CMCNa and HEC mixture was crosslinked with 0.75%; 1.00%; 1.75%; or 2.75% citric acid at 90° C. for 24 hours. The samples were immersed in water for different amounts of time, and the swelling ratio for each sample calculated. As can be seen in FIG. 2, the amount of water absorbed by the hydrogels cross-linked with 0.75%; 1.00%; 1.75%; or 2.75% citric acid at 90° C. for 24 hours increased with the length of time that the sample was immersed in water, and increased as the amount of cross-linker increased. Hydrogels cross-linked with 0.75%; 1%; and 2.75% citric acid at 90° C. for 24 hours became saturated after several hours, while hydrogels cross-linked with 1.75% citric acid at 90° C. for 24 hours did not appear to saturate even after 24 hours. After being immersed in water for 24 hours, hydrogels cross-linked with 0.75% citric acid absorbed about 205 times their own weight of water; hydrogel cross-linked with 1.00% citric acid absorbed about 148 times their own weight of water; hydrogels cross-linked with 1.75% citric acid absorbed only about 139 times their own weight of water; and hydrogels cross-linked with 2.75% citric acid absorbed only about 61 times their own weight of water.

The firmness of the polymer hydrogels cross-linked with citric acid appear to be directly correlated with the amount of citric acid present in the reaction. Polymer hydrogels prepared with CMCNa and HEC with higher concentrations of citric acid (2.75% or 1.75%) were fairly firm, but polymer hydrogels prepared with lower concentrations of citric acid (1.00% or 0.75%) were soft and like a viscous liquid, they were flabby and fairly fragile.

Polymer hydrogels were prepared with CMCNa and HEC, and cross-linked with 1.75% citric acid for 24 hours at different temperatures. As seen in FIG. 3, the temperature of the cross-linking reaction affected gel formation and water absorption. Polymer hydrogels cross-linked with 1.75% citric acid at 70° C. were soft and did not form a physically cohesive material, their swelling ratio was about 210. Hydrogels cross-linked with 1.75% citric acid at 80° C. were slightly flabby and loose, and their swelling ratio reached about 243. Hydrogels cross-linked with 1.75% citric acid at 90° C. were not too firm or too soft, and their swelling ratio was about 138. Hydrogels cross-linked with 1.75% citric acid at 100° C. were very firm, and their swelling ratio dropped sharply to about 13.

The results shown in this Example indicate that by changing the amount of citric acid cross-linker, and the temperature at which the cross-linking reaction occurs, biobased polymer hydrogels with different properties were prepared.

### Example 3

### Swelling Ratio of Polymer Hydrogels Cross-Linked with Succinic Acid or Sebacic Acid

This example shows that the swelling ratio of cellulose-based polymer hydrogels prepared with either succinic acid as cross-linker is similar to the swelling ratios of cellulose-based hydrogels prepared with citric acid. This example also shows that the swelling ratios of cellulose-based hydrogels prepared with sebacic acid follow a different trend than those cross-linked with citric acid or succinic acid.

Hydrogels were prepared with CMCNa and HEC and cross-linked with either 0.75%; 1.00%; 1.75%; or 2.75% succinic acid; or with 1.75%; 2.75%; 3.50%; or 4.25% sebacic acid. The swelling ratios of hydrogels cross-linked for 24 hours at 90° C. were determined.

After immersion in water for 24 hours, the water absorption of the hydrogels cross-linked with succinic acid show similarities to the hydrogels cross-linked with citric acid in Example 2. As seen in FIG. 4, the water absorption of hydrogels cross-linked with succinic acid decreased with increased cross-linker concentrations, which was the same trend seen in FIG. 2 for hydrogels cross-linked with citric acid. After immersion in water for 24 hours, hydrogels cross-linked with 0.75% succinic acid absorbed more than 213 times their own weight of water. This result is similar to the swelling ratio of about 205 obtained with hydrogels cross-linked with 0.75% citric acid. Hydrogels cross-linked with 1.00% succinic acid absorbed more than 152 times their own weight of water. This result is similar to the swelling ratio of about 148 obtained for hydrogels cross-linked with 1.00% citric acid. Hydrogels cross-linked with 1.75%; or 2.75% succinic acid absorbed about half as much water than did hydrogels cross-linked with 1.75%; or 2.75% citric acid. Even after 24 hours immersed in water, none of the hydrogels cross-linked with succinic acid appeared to be saturated.

Hydrogels cross-linked with sebacic acid presented very different water absorption properties when compared with hydrogels cross-linked with citric acid or with succinic acid. No hydrogel was formed when attempting to cross-link with sebacic acid concentrations lower than 1.75%. As seen in FIG. 5, water absorption of hydrogels cross-linked with sebacic acid increased as the concentration of sebacic acid increased. This trend is opposite to the one seen for hydrogels cross-linked with citric acid or succinic acid. The swelling ratio of hydrogels cross-linked with 1.75% sebacic acid was about 110, which is similar to the swelling ratio of hydrogels cross-linked with 2.75% citric acid. After immersion in water for 24 hours, hydrogels cross-linked with 2.75% sebacic acid absorbed more than about 140 times their own weight of water. Hydrogels cross-linked with 3.50% sebacic acid absorbed more than 210 times their own weight of water. Hydrogels cross-linked with 4.25% succinic acid absorbed at least about 260 times their own weight of water. Even after 24 hours immersed in water, none of the hydrogels cross-linked with sebacic acid appeared to be saturated.

### Example 4

### Thermal Decomposition of Polymer Hydrogels of the Invention

This example shows the results of thermogravimetric analyses of the polymer hydrogels of the invention.

As an additional evidence for the cross-linking reaction, thermogravimetric analysis was performed. FIG. x(a, b, and c) show the thermal decomposition of CMCNa, HEC, and their 3:1 mixture as a function of heating temperature. In the case of CMCNa, major decomposition occurred at 250-300° C. and wt. loss at 300° C. is ˜50%. On the other hand, HEC decomposed at 200-350° C. and wt. loss at 350° C. is ˜85%. When CMCNa was mixed with HEC, overall decomposition profile was similar to that of CMCNa as the fraction of HEC was only 25%. After the reaction with each cross-linker, the degradation profile of the CMCNa/HEC mixture was changed dramatically. Unlike single major decomposition of the starting material (see DTG of FIG. x(c)), the cross-linked products showed two major peaks in the DTG (FIG. x(f)). More specifically, the single peak at 290° C. before cross-linking was changed to two peaks at 240 and 300-310° C. There is no doubt that this difference is not due to the degradation of cross-linkers because their fraction is only 1.75% of the total weight of reaction mixture and their thermograms are very much different from each other (FIG. x(d)). The shift of the peak position for the decomposition at 260-330° C. to higher temperature by >20° C. after the reaction proves that cross-linkers formed stronger bonds than before the reaction. The minor DTG peaks shown in FIG. x(f) (i.e., the peaks shown in the inset of FIG. x(e)) are believed to be due to the small amount of gas generated at the beginning of the thermal degradation of cross-linked product; when gas is generated, it exerts pressure on the TGA pan.

The foregoing detailed description and certain representative embodiments and details of the invention have been presented for purposes of illustration and description of the invention. It is not intended to be exhaustive or to limit the invention to the precise forms disclosed. It will be apparent to practitioners skilled in the art that modifications and variations may be made therein without departing from the scope of the invention. All references cited herein are incorporated by reference in their entirety.

We claim:

1. A polymer hydrogel comprising sodium carboxymethyl cellulose (CMCNa) and hydroxyethyl cellulose (HEC) covalently cross-linked with citric acid, succinic acid, or sebacic acid.

2. The polymer hydrogel of claim 1, wherein the polymer hydrogel comprises a 3 to 1 ratio of CMCNa to HEC.

3. The polymer hydrogel of claim 1, wherein the polymer hydrogel is cross-linked with 0.75%; 1.00%; 1.75%; or 2.75% citric acid.

4. The polymer hydrogel of claim 1, wherein the polymer hydrogel is cross-linked with 0.75%; 1.00%; 1.75%; or 2.75% succinic acid.

5. The polymer hydrogel of claim 1, wherein the polymer hydrogel is cross-linked with 1.75%, 2.75%, 3.50%, or 4.255% sebacic acid.

6. The polymer hydrogel of claim 3, wherein the polymer hydrogel can absorb at least about 50 times its dry weight of water at room temperature.

7. The polymer hydrogel of claim 4, wherein the polymer hydrogel can absorb at least about 25 times its dry weight of water at room temperature.

8. The polymer hydrogel of claim 5, wherein the polymer hydrogel can absorb at least about 80 times its dry weight of water at room temperature.

9. A soil conditioning element, a soil nutrient carrier, a soil erosion reduction element, or a water infiltration soil additive comprising a polymer hydrogel of claim 1.

10. A contact lens, a hygiene product, a tissue engineering scaffold, a drug delivery system, a wound dressing, a cell culture well-coating, a biosensor, a medical electrode, or a water gel explosive comprising a polymer hydrogel of claim 1.

11. A pharmaceutical composition comprising a polymer hydrogel of claim 1, and optionally a pharmaceutically-acceptable carrier.

12. The pharmaceutical composition of claim 11, in the form of a tablet or a capsule.

13. A method for preparing a polymer hydrogel, the method comprising:
mixing CMCNa, HEC, and citric acid, succinic acid, or sebacic acid to form a reaction solution;
concentrating the reaction solution by heating to remove water; and
heating the concentrated reaction solution to cross-link the hydrogel.

14. The method of claim 13, wherein the CMCNa and HEC are in a 3:1 ratio.

15. The method of claim 13, wherein citric acid or succinic acid are added at 0.75%; 1.00%; 1.75%; or 2.75%.

16. The method of claim 13, wherein sebacic acid is added at 1.75%; 2.75%; 3.50%; or 4.25%.

17. The method of claim 13, wherein concentrating the reaction solution by heating to remove water is performed by heating for at least about 8 hours to about 18 hours.

18. The method of claim 17, wherein heating to remove water is performed at 55° C.

19. The method of claim 13, wherein the concentrated solution is heated at 70° C.; 80° C.; 90° C.; or 100° C.
